(19) United States
(12) Patent Application Publication
(10) Pub. No.: US 2012/0096570 A1
(43) Pub. Date: Apr. 19, 2012

(54) BASE FOR PROPAGATING AQUATIC ANIMALS

(76) Inventors: Tin Tran, Crowley, TX (US); Nicholas Nevid, Crowley, TX (US)

(21) Appl. No.: 12/906,319

(22) Filed: Oct. 18, 2010

(51) Int. Cl. A01K 67/00; A01K 61/00

(52) U.S. Cl. 800/8; 119/200

(57) ABSTRACT

An aquatic animal such as coral is propagated on a base. The base has a top portion with a groove located therein. The groove has converging side walls. The groove intersects a depression that extends from the top portion to a bottom portion of the base. The depression has converging sides. An animal fragment is secured to the base by locating it in the groove, or alternatively in the groove and the depression. Water flow across the base may cause the fragment to move inside of the groove, wherein the fragment will sink down and become wedged in place, further securing or coupling the fragment to the base. The coral then grows and adheres itself to the base.

### FIELD OF THE INVENTION

The present invention relates to a base or support for propagating aquatic animals, such as coral or sponges.

### BACKGROUND OF THE INVENTION

Corals are a diverse group of anemone-like animals that inhabit marine environments. Corals include soft corals, hard corals, sponges, sea fans, etc. Corals are grown and sold commercially for use in aquariums that are located in homes, businesses and public aquariums. Corals are also grown for restocking reefs.

Many types of coral can be grown asexually by propagation from fragments or “frags”. For example, a piece of living coral can be broken into smaller pieces or fragments. A fragment is then secured to a base or support. The base typically has a depression or hole in its top for receiving the coral fragment.

As the fragment of coral grows, it enlarges and gradually adheres to the base. When the coral is grown sufficiently large, the coral, together with its base, are harvested for sale. The base becomes part of the coral. When the coral is purchased by a user, the coral and its base are put into an aquarium of water.

Securing the coral fragment to the base during the initial stages of propagation is important in order for the coral to grow correctly. The base and the coral fragment are put into a tank or natural body of water where the water flows over the coral, delivering nutrients and food. If the coral is improperly secured to the base, then the coral fragment will rock or move and attachment of the coral to the base will be delayed or not occur. This may stunt the growth of the coral and make it difficult to handle and transport for shipment at a later time.

In the prior art, there are several ways to secure the coral fragments to the base. One way uses an adhesive in the bottom of the depression, which depression is located in the top of the base. The coral fragment is inserted into the adhesive. U.S. Patent Publication No. 20050022749 illustrates this technique. Another way involves using rubber bands that wrap around the coral fragment on top of the base and around the bottom of the base. Still other methods employ a string or fishing line.

Both of these methods add to the expense of coral propagation and are labor intensive. For the adhesive method, an amount of adhesive must be applied to the base, followed by locating a fragment of coral in the adhesive.

For the rubber band method, the coral is placed on the base and the band is wrapped around the coral and the base. After the coral has grown large enough to attach itself to the base, the rubber band is removed to prevent the coral from incorporating the portion of the rubber band in contact with the coral into its tissues. Trying to time the removal of the rubber band from the coral is difficult. In addition, from time to time, the rubber band will break, thus failing to secure the coral to the base. If the coral has attached itself to the rubber band already and the rubber band breaks, then the coral may be propelled away from its base (across, or even outside of, the tank of water) by the power of the snapping rubber band.

Thus, it is desired to reduce the amount of labor involved in attaching coral to a base, keeping the coral in place and in obtaining a strong and aesthetic bond between the coral and its base.

### SUMMARY OF THE INVENTION

The present invention provides a base for propagation of aquatic animals comprising a bottom portion and a top portion. A groove is formed in the top portion. The groove has side walls that converge toward the bottom portion. The groove has a length and a width. The length is at least one and one fourth the distance of the width.

In accordance with another aspect of the present invention, a depression is located in the top portion and extends toward the bottom portion. The depression intersects the groove. The depression has side walls that converge toward the bottom portion.

In accordance with another aspect of the present invention, the depression extends through the base to allow water to circulate therethrough.

In accordance with still another aspect of the present invention, the groove intersects a center of the upper portion of the depression. The groove has an end on opposite sides of the depression.

In accordance with still another aspect, sides are between the top and bottom portions. The top has a mound shape.

In accordance with still another aspect, the groove has a depth that is greater than the width.

In accordance with another aspect, a depression is located in the top portion and extends toward the bottom portion. The depression intersects the groove. The depression has side walls that converge toward the bottom portion. The depression extends through the base to allow water to circulate therethrough. The groove intersects a center of the upper portion of the depression. The groove has an end on opposite sides of the depression. The base has sides between the top and bottom portions. The base has a mound shape.

The present invention also provides a propagated, immobile aquatic animal, comprising a base and the animal. The base comprises a bottom portion and a top portion. The base has a groove formed in the top portion. The groove has side walls that converge toward the bottom portion. The groove has a length and a width. The length is at least twice the distance of the width. The aquatic animal is located in the groove and is coupled to the base by the growth of the animal.

In accordance with one aspect of the invention, the base further comprises a depression located in the top portion and extending toward the bottom portion. The depression intersects the groove. The depression has side walls that converge toward the bottom portion.

In accordance with still another aspect, the depression extends through the base to allow water to circulate therethrough.

In accordance with still another aspect, the groove intersects a center of the upper portion of the depression. The groove has an end on opposite sides of the depression.

In accordance with still another aspect, sides extend between the top and bottom portions of the base. The base has a mound shape.

In accordance with still another aspect, the groove has a depth that is greater than the width.

In accordance with still another aspect, a depression is located in the top portion and extends toward the bottom portion. The depression intersects the groove. The depression has side walls that converge toward the bottom portion. The depression extends through the base to allow water to circulate therethrough. The groove intersects a center of an upper portion of the depression. The groove has an end on opposite sides of the depression. Sides extend between the top and bottom portions. The base had a mound shape.

The present invention also provides a method of propagating an aquatic animal. A base is provided having a bottom surface and a top portion. The base has a groove located in the top portion. The groove has converging side walls. The groove has a length and a width. The length is at least twice the distance of the width. A fragment of the animal is obtained. The fragment is secured to the base by locating the fragment in the groove. The base is placed on its bottom surface and water.

In accordance with still another aspect of the invention, the step of providing a base further comprises providing a base with a depression that intersects the groove. The depression has converging side walls. The step of securing the fragment further comprises locating the fragment in the depression and in the groove.

### BRIEF DESCRIPTION OF THE DRAWINGS

FIG. 1 is an isometric view of the base, in accordance with a preferred embodiment.

FIG. 2 is a cross-sectional view of the base, taken at lines II-II of FIG. 2, showing a coral fragment located therein and the base located on a support.

FIG. 3 is a plan view of the base, showing a coral fragment therein.

FIG. 4 is a cross-sectional view of the base, taken at lines IV-IV of FIG. 1, showing a different type of coral fragment located therein.

FIG. 5 is cross-sectional view of the base, taken along the orientation lines IV-IV of FIG. 1, showing a fragment of a different type.

FIG. 6 is a side view of the base of FIG. 4 shown after the coral has grown and is ready for harvesting.

FIG. 7 is cross-sectional view of the base of FIG. 5, showing a grown coral ready for harvesting.

### DESCRIPTION OF THE PREFERRED EMBODIMENTS

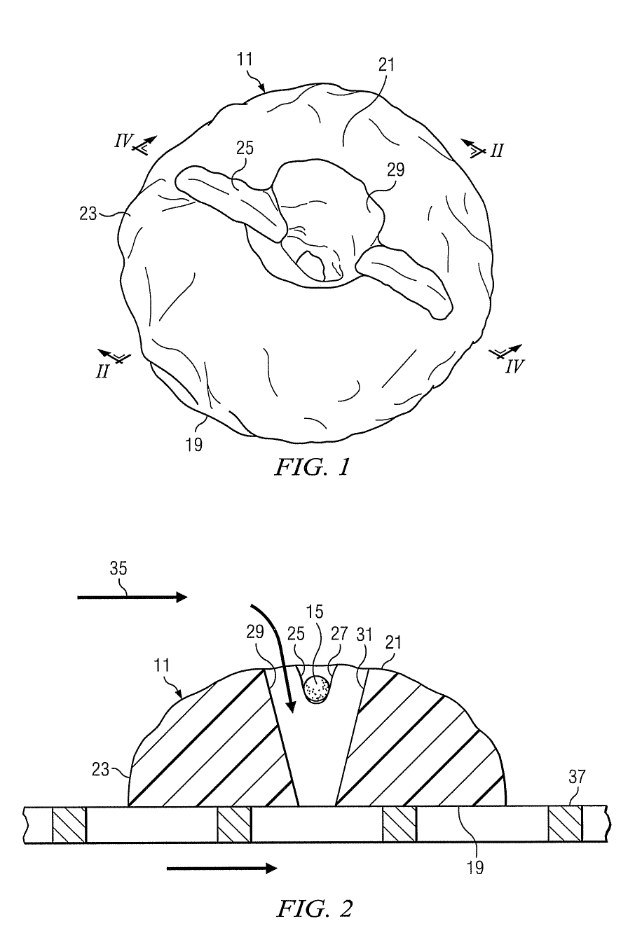

The base 11 (see FIGS. 1 and 2) described herein is used to propagate aquatic animals 15 by providing a foundation or support to allow an animal to grow. Once grown, as shown in FIG. 6, the animal and base are coupled together to form a single immobile unit 17 that can be sold for use in an aquarium or used to restock reefs. The base is thus used in the practice of aquaculture.

In the description that follows, terms such as “top” or “bottom” will be used with reference to the orientation shown in the drawings.

The animal 15 is an aquatic animal and is typically of the Cnidaria (such as corals) or Porifera (such as sponges) phyla. There are a wide variety of corals and sponges that can be used in conjunction with the base 11.

Because the base is sold together with the coral as a single unit, the base 11 preferably emulates a natural support such as a rock. The base 11 has a bottom portion 19 that includes a preferably flat bottom surface. A flat surface needs no special equipment and is universally accepted by both the aquiculturalists who grow the coral and the ultimate purchaser who places the coral in an aquarium. The base has a top portion 21. Because the base emulates a rock, the sides 23 slope down from the top portion to the bottom portion.

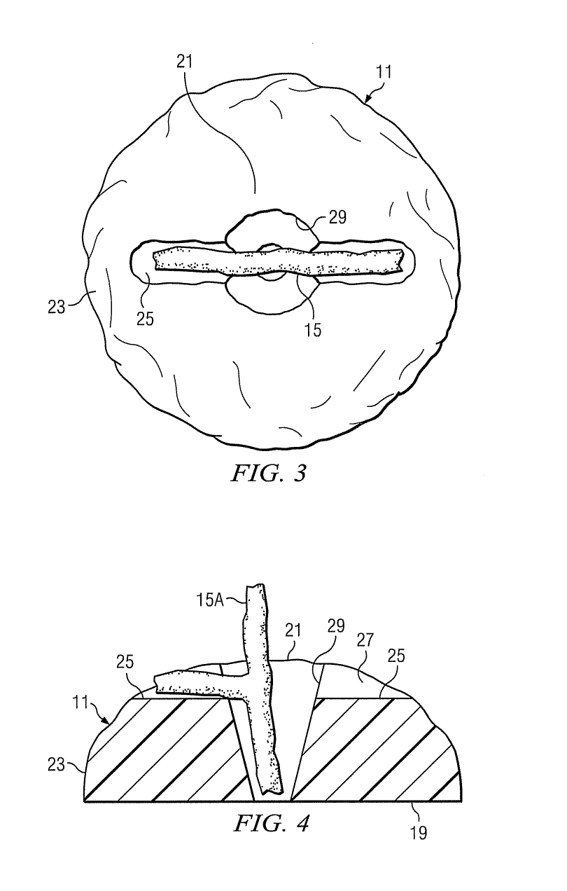

The top portion 21 has a groove 25 or notch located therein. The groove has a length and a width. Referring to the orientation shown in FIG. 3, the length is the left to right dimension, while the width is the up to down dimension. The fragment 15 is shown lying along the length of the groove. The length of the groove is at least one and one fourth (1¼) the width of the groove. Alternatively, the length of the groove is at least twice the width. In the examples shown, the length is six or seven times the width of the groove.

The groove 25 is relatively deep and narrow. The depth of the groove is greater than the width. Preferably, the depth is 1.5 to 2 times the width of the groove. The sides 27 of the groove 25 slope inwardly or converge from the top toward the bottom of the base. This gives the groove an approximate “V” shape as shown in FIG. 2. The bottom of the groove need not be pointed as in a “V” but can be rounded as in a “U” shape.

The base can have more than one groove. For example, two grooves in an “X” configuration can be used, but because it is desirable for the coral to grow and fill in the groove, a single groove is preferred.

The base 11 also has a hole 29 or depression that extends from the top portion down. In the preferred embodiment, the hole 29 extends all the way to the bottom surface 19 so that water and debris can exit through the hole. The hole 29 intersects the groove 25. The hole has tapering sides 31 that converge from the top portion to the bottom.

In the preferred embodiment, the groove 25 and hole 29 are centered on the top portion 21 of the base for aesthetic reasons. The hole is centered along the groove so that there is an end of the groove on each side of the hole. However, either the groove, the hole or both can be located off center.

The base 11 can be made of a variety of materials such as cement, plastic, etc. The base can be molded.

The base is preferably, but need not be, aesthetic. When the coral grows, the base will be visible. The base is mound shaped for aesthetic appearance, such as shown in the drawings, with a wide bottom and rounded top and sides. The base as shown emulates a rock and has a natural appearance, with the sides and top portion being irregular or rough. The base can be of various sizes and shapes, and also various heights. The top could be flat or even concave.

To use, a fragment 15 of an animal is obtained. If the animal is hard, this involves taking a large piece and breaking it into smaller fragments. If the animal is soft, then a knife or pair of scissors can be used to cut the fragment off. If the coral is branch-like, the fragment will be like a small stick, with a roughly cylindrical shape, and possibly having side branches. The fragment is located in the groove 25 as shown in FIGS. 2 and 3, where the fragment extends across the top of the hole 29 and with the fragment ends located in the groove. Preferably, the fragment is gently pushed into the groove so as to wedge the fragment inside the groove. This provides immediate securing or coupling of the coral to the base. If the fragment is easily damaged, then it need not be pushed into the groove, but is simply located therein. No further steps are needed to secure the fragment to the base.

The base and fragment are then located in a tank of water on a support 37 that is typically perforated. Water 35 flows over and under the base, delivering nutrients and food to the coral. Water can also flow through the hole 29. The groove is wedge shaped in cross-section. As water flows over the base and the fragment 15, the fragment 15 may rock inside of the groove 25. As it does so, the fragment falls deeper into the groove and becomes wedged in place. Thus, the natural action or movement of the fragment serves to further secure it in place to the base.

FIG. 4 shows another shape of fragment 15A, one with a change of direction, like an “L” or “Y”. One part of the coral is located in the hole 29, with another part located in the groove 25. Once again, any motion by the fragment causes it to work itself under the groove or opening and become wedged in place.

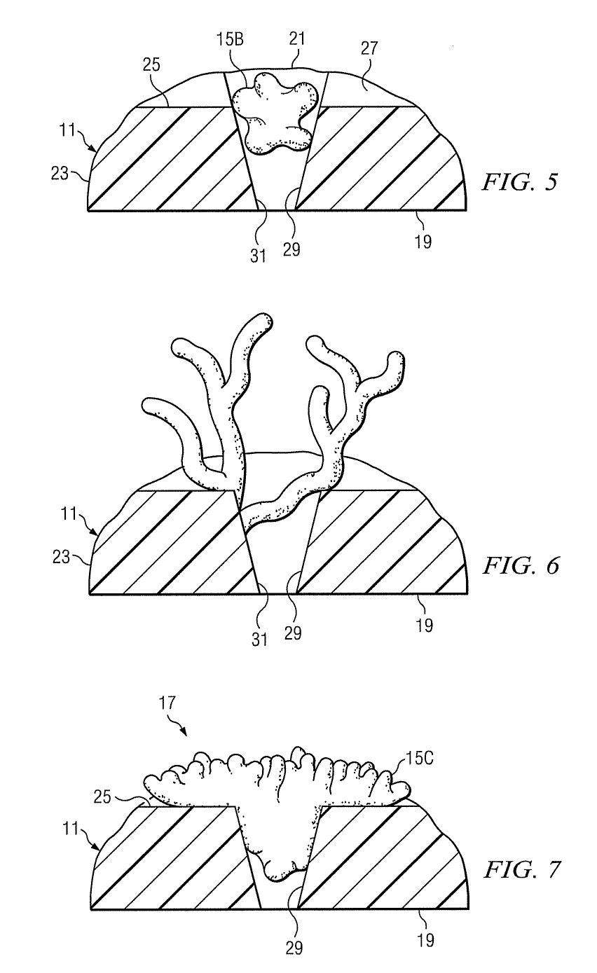

FIG. 5 shows another type of fragment 15B, one that is like a button. The fragment is located in the opening or hole 29, where it becomes wedged in place. As the coral 15C grows, it enters the groove and will cover the hole, as shown in FIG. 6.

The frag need not be placed in the groove. For example, if the frag has side branches, it can be located in the hole 29 without being located in the groove. Thus, the base is highly versatile.

The base and coral unit are particularly aesthetic and desirable because as the coral grows, it is not located down in a deep depression or cavity inside the base. Such a deeply placed fragment of coral encounters problems. As the coral grows, bacteria, sand or other debris may enter the depression before the coral has a chance to grow and fill in the depression. This allows dirty and infectious material to contaminant the coral specimen and is both unsightly and may serve to introduce an infection into an aquarium. Also, it is desirable to have the coral fill the cavity completely to provide a good, strong bond with the base. If foreign matter is present in the depression, then the coral grows around it, producing a weak connection to the base. As the coral grows to a larger size, the weak coupling to the base may fail, wherein the coral breaks off the base and falls to the bottom of the aquarium.

Implanting a fragment in the base is very simple and requires less labor than with the prior art. The fragment is simply located in the groove or hole. No additional steps, such as providing adhesives or wrapping with a rubber band, are required. As a result, coral can be grown less expensively and enables the artificial propagation of corals. As a practical matter, this means that coral need not be harvested from the open ocean and imported, making coral a sustainable resource.

The foregoing disclosure and showings made in the drawings are merely illustrative of the principles of this invention and are not to be interpreted in a limiting sense.

1. A base for propagation of aquatic animals, comprising:
a) a bottom portion;
b) a top portion;
c) a groove formed in the top portion, the groove having side walls that converge toward the bottom portion, the groove having a length and a width, the length being at least one and one fourth that of the width.

2. The base of claim 1, further comprising a depression located in the top portion and extending toward the bottom portion, the depression intersecting the groove, the depression having side walls that converge toward the bottom portion.

3. The base of claim 2, wherein the depression extends through the base to allow water to circulate therethrough.

4. The base of claim 2, wherein the groove intersects a center of an upper portion of the depression with the groove having an end on opposite sides of the depression.

5. The base of claim 1, further comprising sides between the top and bottom portions, the base having a mound shape.

6. The base of claim 1 wherein the groove has a depth that is greater than the width.

7. The base of claim 1, further comprising:
a) a depression located in the top portion and extending toward the bottom portion, the depression intersecting the groove, the depression having side walls that converge toward the bottom portion;
b) the depression extends through the base to allow water to circulate therethrough;
c) the groove intersects a center of an upper portion of the depression with the groove having an end on opposite sides of the depression;
d) sides between the top and bottom portions, the base having a mound shape.

8. A propagated, immobile aquatic animal, comprising:
a) a base comprising a bottom portion and a top portion, the base having a groove formed in the top portion, the groove having side walls that converge toward the bottom portion, the groove having a length and a width, the length being at least twice that of the width;
b) the aquatic animal located in the groove and coupled to the base by the growth of the animal.

9. The animal of claim 8, wherein the base further comprises a depression located in the top portion and extending toward the bottom portion, the depression intersecting the groove, the depression having side walls that converge toward the bottom portion.

10. The animal of claim 9, wherein the depression extends through the base to allow water to circulate therethrough.

11. The animal of claim 9, wherein the groove intersects a center of an upper portion of the depression with the groove having an end on opposite sides of the depression.

12. The animal of claim 8, further comprising sides between the top and bottom portions, the base having a mound shape.

13. The animal of claim 8, further comprising:
a) a depression located in the top portion and extending toward the bottom portion, the depression intersecting the groove, the depression having side walls that converge toward the bottom portion;
b) the depression extends through the base to allow water to circulate therethrough;
c) the groove intersects a center of an upper portion of the depression with the groove having an end on opposite sides of the depression;
d) sides between the top and bottom portions, the base having a mound shape.

14. The animal of claim 8, wherein the groove has a depth that is greater than the width.

15. A method of propagating an aquatic animal, comprising the steps of:
a) providing a base having a bottom surface and a top portion, the base having a groove located in the top portion, the groove having converging side walls, the groove having a length and a width, the length being at least twice that of the width;
b) obtaining a fragment of the animal;
c) securing the fragment to the base by locating the fragment in the groove;
d) placing the base on its bottom surface in water.

16. The method of claim 15, wherein:
a) the step of providing a base further comprises providing a base with a depression that intersects the groove, the depression has converging side walls;
b) the step of securing the fragment further comprises the step of locating the fragment in the depression and in the groove.
